(19) United States
(12) Patent Application Publication
(10) Pub. No.: US 2024/0233919 A1
(43) Pub. Date: Jul. 11, 2024

(54) FAST MEDICAL HYPERSPECTRAL IMAGE (MHSI) CLASSIFICATION METHOD BASED ON SIMILARITY TANGENT MAPPING

(71) Applicant: Shandong University, Jinan City (CN)

(72) Inventors: Xiaopeng MA, Jinan City (CN); Chenglong ZHANG, Jinan City (CN); Dexin YU, Jinan City (CN); Xiaoli YANG, Jinan City (CN); Qiyuan CHENG, Jinan City (CN)

(21) Appl. No.: 18/405,293

(22) Filed: Jan. 5, 2024

(30) Foreign Application Priority Data
Jan. 6, 2023 (CN) 202310016620.3

(51) Int. Cl. G16H 30/40; G06T 7/00

(52) U.S. Cl. CPC G16H 30/40; G06T 7/0012; G06T 2207/30024

(57) ABSTRACT

The present disclosure provides a fast medical hyperspectral image (MHSI) classification method based on similarity tangent mapping, and relates to the technical field of hyperspectral image (HSI) processing. The fast MHSI classification method includes: preprocessing a to-be-classified MHSI; extracting a sample set from the to-be-classified MHSI; dividing the sample set into a training sample set and a test sample set; constructing a cosine similarity tangent mapping (CSTM) model based on the training sample set; and inputting the test sample set into the CSTM model to obtain a classification result. Tangent mapping is performed for a cosine similarity to evaluate a similarity between pixels, and a similarity between a to-be-classified pixel and each different type of training sample is calculated based on a joint local region. Afterwards, a category label of the to-be-classified pixel is allocated based on the similarity to quickly obtain a stable classification result.

### CROSS REFERENCE TO RELATED APPLICATION

This patent application claims the benefit of and priority to Chinese Patent Application No. 202310016620.3, filed with the Chinese patent office on Jan. 6, 2023, which is hereby incorporated by reference herein in its entirety.

### TECHNICAL FIELD

The present disclosure relates to the technical field of hyperspectral image (HSI) processing, and specifically, to a fast medical hyperspectral image (MHSI) classification method based on similarity tangent mapping.

### BACKGROUND

Compared with a traditional color digital image, a hyperspectral image (HSI) has higher spectral resolution, typically containing tens to hundreds of bands. Rich spectral information can provide a basis for accurately identifying a target. Therefore, the HSI is widely used in the field of remote sensing. With the advancement of science and technology, advantages of spectral imaging have been applied in various fields, for example, archaeological mural protection, physical evidence identification, and non-destructive testing of food. With the continuous development of the medical spectral imaging technology, medical health has become a fastest-growing application field of the HSI.

For medical applications, a medical hyperspectral image (MHSI) can not only provide two-dimensional spatial distribution information of various tissue structures, but also obtain a complete spectrum of a point on a biological tissue sample in an interested wavelength range to analyze chemical compositions and physical characteristics of different pathological tissues. Therefore, fast and accurate MHSI classification makes it possible to perform non-invasive disease diagnosis and achieve clinical treatment applications.

MHSI classification is to allocate a semantic label to a pixel based on a feature of an image. In early research on HSI classification, some classifiers based on spectral information such as a support vector machine (SVM) classifier, a k-nearest neighbor (KNN) classifier, and a sparse representation (SR) classifier have achieved certain success. In recent years, in order to make full use of a spatial feature of the HSI, many classification methods based on spatial-spectral features, such as joint sparse representation (JSR) classification, joint nearest neighbor (JNN) classification, and joint intraclass collaborative representation (JCR) classification, have obtained high-precision classification results based on spatial neighborhood information of the pixel.

However, due to lack of utilization of spatial contextual information, a classification method based on the spectral information often results in a large number of noise spots in a classification result, making it difficult to meet an application demand of the HSI. For an ultra-complex surface, especially when a to-be-classified pixel is in a heterogeneous region, distinguishing performance of a current method based on spatial-spectral information fusion is degraded due to interference from a heterogeneous pixel, and this type of method usually requires longer computation time due to the spatial-spectral information fusion.

### SUMMARY

In view of this, embodiments of the present disclosure provide a fast MHSI classification method based on similarity tangent mapping, to resolve a prior-art problem that an existing method based on spatial-spectral information fusion has degraded distinguishing performance due to interference from a heterogeneous pixel and long computation time.

An embodiment of the present disclosure provides a fast MHSI classification method based on similarity tangent mapping, including:
preprocessing a to-be-classified MHSI;
extracting a sample set from the to-be-classified MHSI;
dividing the sample set into a training sample set and a test sample set;
constructing a cosine similarity tangent mapping (CSTM) model based on the training sample set; and
inputting the test sample set into the CSTM model to obtain a classification result.

Optionally, the preprocessing a to-be-classified MHSI includes:
obtaining a pixel quantity, a spectral dimension, and a tissue category of the to-be-classified MHSI; and
normalizing, band by band, a spectral value corresponding to each pixel.

Optionally, the extracting a sample set from the to-be-classified MHSI includes:
extracting a sample quantity and a tissue category label of each sample.

Optionally, the constructing a CSTM model based on the training sample set includes:
calculating a cosine similarity between a to-be-classified pixel and each training sample in feature space to constitute a cosine similarity matrix;
performing tangent mapping for the cosine similarity matrix;
calculating a similarity between the to-be-classified pixel and a training sample of each different tissue category by combining a spatial neighborhood; and
allocating a label to the to-be-classified pixel based on a highest similarity.

Optionally, the calculating a similarity between the to-be-classified pixel and a training sample of each different tissue category by combining a spatial neighborhood includes:
obtaining a pixel and a neighborhood scale within the spatial neighborhood of the to-be-classified pixel;
obtaining a highest similarity between the to-be-classified pixel and each type of sample based on the cosine similarity matrix;
representing a similarity between the to-be-classified pixel and each type of sample by using an average similarity of all neighborhood pixels; and
comparing similarities between the to-be-classified pixel and different tissue categories, and adding the to-be-classified pixel to a tissue category with a highest similarity.

Optionally, the obtaining a pixel and a neighborhood scale within the spatial neighborhood of the to-be-classified pixel includes:
obtaining an optimal neighborhood window scale through k iterative cross-validation operations.

Optionally, the normalizing, band by band, a spectral value corresponding to each pixel includes:
obtaining a first difference between the pixel and a minimum spectral value of a corresponding band;
obtaining a second difference between a maximum spectral value of the corresponding band of the pixel and the minimum spectral value; and
normalizing the spectral value of the pixel into a ratio of the first difference to the second difference.

Optionally, the obtaining an optimal neighborhood window scale through k iterative cross-validation operations includes:
randomly dividing training sample data into k equal fractions;
taking turns to use k−1 fractions of data as training data and remaining one fraction of data as test data;
taking an average value of iterative crossover results as estimation accuracy; and
taking a neighborhood window scale corresponding to highest accuracy as the optimal neighborhood window scale.

The embodiments of the present disclosure have following beneficial effects:

The embodiments of the present disclosure provide a fast MHSI classification method based on similarity tangent mapping. Tangent mapping is performed for a cosine similarity to evaluate a similarity between pixels, and a similarity between a to-be-classified pixel and each different type of training sample is calculated based on a Joint local region. The cosine similarity undergoing the tangent mapping can effectively reduce interference from a heterogeneous pixel, thereby ensuring full utilization of spatial-spectral information of the MHSI. Afterwards, a category label of the to-be-classified pixel is allocated based on the similarity to quickly obtain a stable classification result.

### BRIEF DESCRIPTION OF THE DRAWINGS

The features and advantages of the present disclosure can be more clearly understood with reference to the accompanying drawings. The accompanying drawings are illustrative and should not be understood as any limitation on the present disclosure. In the accompanying drawings:

FIG. 1 is a flowchart of a fast MHSI classification method based on similarity tangent mapping according to an embodiment of the present disclosure;

FIG. 2 is a flowchart of another fast MHSI classification method based on similarity tangent mapping according to an embodiment of the present disclosure;

FIG. 3 is a flowchart of a classification method based on a CSTM model according to an embodiment of the present disclosure;

FIG. 4 is a schematic diagram of a curve indicating that classification accuracy varies with a window scale according to an embodiment of the present disclosure;

FIG. 5 shows a false color image of a living tissue of a brain cancer according to an embodiment of the present disclosure;

FIG. 6 shows a real label of a living tissue of a brain cancer according to an embodiment of the present disclosure;

FIG. 7 shows a result of SVM classification of a living tissue of a brain cancer according to an embodiment of the present disclosure;

FIG. 8 shows a result of JNN classification of a living tissue of a brain cancer according to an embodiment of the present disclosure;

FIG. 9 shows a result of JSR classification of a living tissue of a brain cancer according to an embodiment of the present disclosure; and

FIG. 10 shows a result of CSTM classification of a living tissue of a brain cancer according to an embodiment of the present disclosure.

### DETAILED DESCRIPTION OF THE EMBODIMENTS

In order to make the objectives, technical solutions, and advantages of the embodiments of the present disclosure clearer, the technical solutions in the embodiments of the present disclosure will be clearly and completely described below in conjunction with the accompanying drawings in the embodiments of the present disclosure. Apparently, the described embodiments are some, rather than all of the embodiments of the present disclosure. All other embodiments obtained by those skilled in the art based on the embodiments of the present disclosure without creative efforts should fall within the protection scope of the present disclosure.

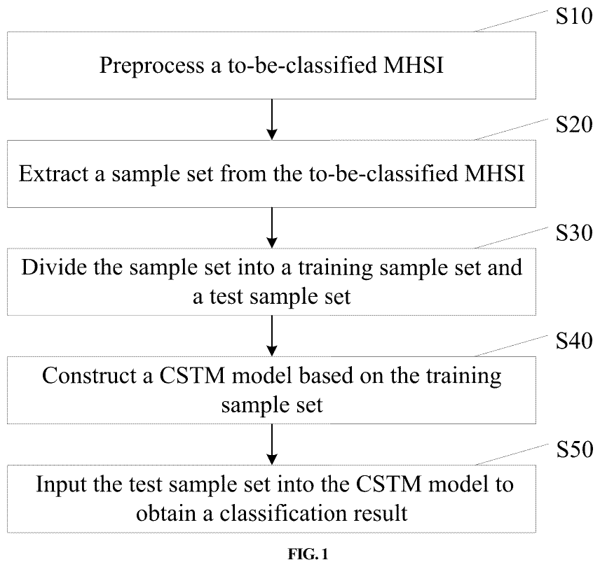

An embodiment of the present disclosure provides a fast MHSI classification method based on similarity tangent mapping. As shown in FIG. 1, the fast MHSI classification method includes following steps.

Step S10: Preprocess a to-be-classified MHSI.

In this embodiment, a pixel quantity, a spectral dimension, and a tissue category of the to-be-classified MHSI are obtained, and a spectral value corresponding to each pixel is normalized band by band. Specifically, the to-be-classified MHSI Y=[y₁, y₂, . . . , yₙ]∈ [symbol] ^{n×d} is selected, where [symbol] represents a real number field, n represents the pixel quantity of the MHSI, and d represents the spectral dimension.

The normalization includes: obtaining a first difference between the pixel and a minimum spectral value of a corresponding band;
obtaining a second difference between a maximum spectral value of the corresponding band of the pixel and the minimum spectral value; and
normalizing the spectral value of the pixel into a ratio of the first difference to the second difference.

In a specific embodiment, a pixel value in each spectral band of the MHSI is linearly normalized. A normalization process for a spectral value corresponding to a b^{th} pixel in an a^{th} band of the MHSI is as follows:

$${y}_{b , a}^{′} = \frac{{y}_{b , a} - min}{max - min} ,$$

where min and max respectively represent minimum and maximum spectral values in the a^{th} band, and both a and b are real numbers greater than 0.

Step S20: Extract a sample set from the to-be-classified MHSI.

In this embodiment, a sample quantity and a tissue category label of each sample are extracted. In a specific embodiment, the sample set X=[x₁^{l}^{₁}, x₂^{l}^{₂}, . . . , xₘ^{l}^{ₘ}]∈ [symbol] ^{m×d} is extracted from the MHSI based on an actual tissue category, where m represents the sample quantity, lₜ={1, 2, . . . , c} represents a label of a t^{th} sample, c represents a number of classes of the MHSI, and t∈[1,m].

Step S30: Divide the sample set into a training sample set and a test sample set.

In this embodiment, five to ten training sample sets are set.

Step S40: Construct a CSTM model based on the training sample set.

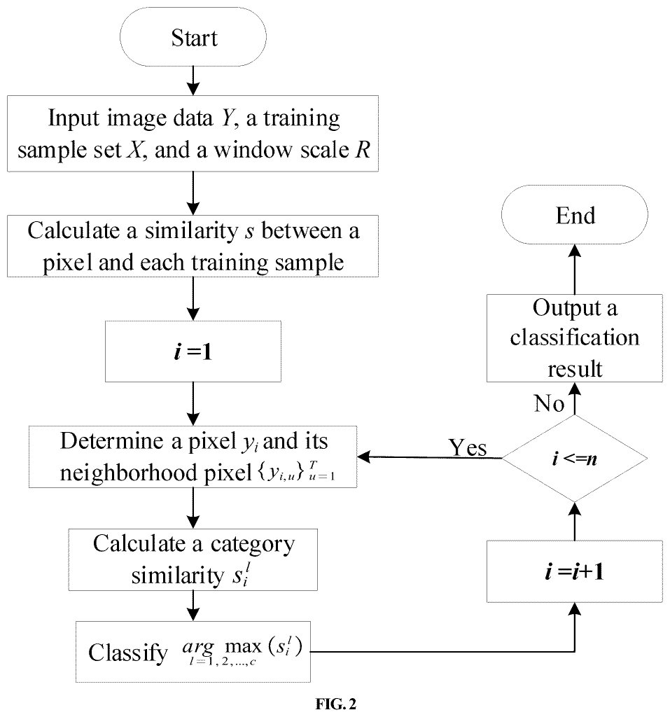

In this embodiment, as shown in FIG. 2, the constructing a CSTM model includes following steps:
S401: Initialize a neighborhood window scale.
S402: Classify the MHSI based on CSTM.
S403: Optimize the window scale through cross-validation.

The steps S402 and S403 are repeated until an optimal window scale is obtained.
S405: Evaluate classification accuracy.
S406: Output a classification map and its corresponding classification accuracy.

Step S50: Input the test sample set into the CSTM model to obtain a classification result.

In this embodiment, the CSTM model is used to classify the original MHSI.

This embodiment of the present disclosure provides a fast MHSI classification method based on similarity tangent mapping. Tangent mapping is performed for a cosine similarity to evaluate a similarity between pixels, and a similarity between a to-be-classified pixel and each different type of training sample is calculated based on a joint local region. The cosine similarity undergoing the tangent mapping can effectively reduce interference from a heterogeneous pixel, thereby ensuring full utilization of spatial-spectral information of an MHSI. Afterwards, a category label of the to-be-classified pixel is allocated based on the similarity to quickly obtain a stable classification result.

As an optional implementation, the step S40 further includes:
calculating a cosine similarity between a to-be-classified pixel and each training sample in feature space to constitute a cosine similarity matrix.

In this embodiment, the cosine similarity between the pixel and each training sample in the feature space is calculated to constitute the similarity matrix s∈ [symbol] ^{n×m}. A cosine similarity between a pixel yᵢ and a training sample pixel xⱼ is calculated as follows:

$${s}_{i , j} = \frac{{y}_{i} \cdot {x}_{j}}{{‖ {y}_{i} ‖}_{2} {‖ {x}_{j} ‖}_{2}}$$

Tangent mapping is performed for the cosine similarity matrix, where both i and j are natural numbers.

In this embodiment, the tangent mapping is performed for the obtained cosine similarity matrix s as follows:

$$s = tan ( \frac{π}{2} \times s - ε )$$

A small constant E is set to prevent an infinite value for an element in the s, and E is set to 10⁻⁶.

A similarity between the to-be-classified pixel and a training sample of each different tissue category is calculated by combining a spatial neighborhood.

In this embodiment, a pixel and a neighborhood scale within the spatial neighborhood of the to-be-classified pixel are obtained. A highest similarity between the to-be-classified pixel and each type of sample is obtained based on the cosine similarity matrix. A similarity between the to-be-classified pixel and each type of sample is represented by using an average similarity of all neighborhood pixels. Similarities between the to-be-classified pixel and different tissue categories are compared, and the to-be-classified pixel is added to a tissue category with a highest similarity.

In a specific embodiment, a similarity between the pixel yᵢ and a category l can be represented by an average similarity of all pixels in a neighborhood of the pixel yᵢ. For the pixel yᵢ, pixels {y_{i,1}, y_{i,2}, . . . , y_{i,u}}T=R² in a spatial neighborhood of the pixel yᵢ are first determined, where R represents the neighborhood scale. Then, a highest similarity Sᵢᵤ^{l} between y_{i,u} and each sample of the category l is obtained based on the similarity matrix s. Finally, the average similarity of all the neighborhood pixels is used to represent the similarity between the yᵢ and the category l:

$${s}_{i}^{l} = \frac{{\sum }_{u = 1}^{T} {s}_{iu}^{l}}{T}$$

A label is allocated to the to-be-classified pixel based on the highest similarity.

In this embodiment, similarities between the yᵢ and different categories are compared, and the yᵢ is added to a category with a highest similarity, that is:

$$class ( {y}_{i} ) = {arg max}_{l = 1 , 2 , … , c} ( {s}_{i}^{l} )$$

$${\{ {y}_{i , u} \}}_{u = 1}^{T}$$

In order to avoid inaccurate expression of a similarity between pixels due to insufficient utilization of spatial information of the MHSI, low computational efficiency of a traditional classification method, and the like, this embodiment of the present disclosure uses the CSTM as a similarity measure between samples based on the cosine similarity, and then calculates similarities between the to-be-classified pixel and different categories based on a joint local region. In this way, spatial-spectral information of an HSI is fully utilized and the to-be-classified pixel is allocated to the category with the highest category. The similarity tangent mapping method provided in the present disclosure can effectively increase aggregation of a homogeneous pixel and separability of a heterogeneous pixel, thereby obtaining a better classification result than the traditional classification method.

As an optional implementation, the obtaining a pixel and a neighborhood scale within the spatial neighborhood of the to-be-classified pixel includes:
obtaining the optimal neighborhood window scale through k iterative cross-validation operations;
randomly dividing training sample data into k equal fractions;
taking turns to use k−1 fractions of data as training data and remaining one fraction of data as test data;
taking an average value of iterative crossover results as estimation accuracy; and
taking a neighborhood window scale corresponding to highest accuracy as the optimal neighborhood window scale.

In this embodiment, the sample data is randomly divided into the k equal fractions, and the k−1 fractions of data are used for training and the one fraction of data is used for testing in turn. An average value of k results is taken as the estimated accuracy to evaluate the R. Generally, 10-fold cross-validation is performed for 10 times to obtain the optimal R.

In a specific implementation, the fast MHSI classification method further includes: evaluating accuracy of the classification result based on a test sample. A remaining labeled sample for training is used to test and calculate a confusion matrix to obtain overall accuracy (OA) and a Kappa coefficient of classification. In this embodiment, classification accuracy and standard deviations of 10 random selections of the training set are recorded.

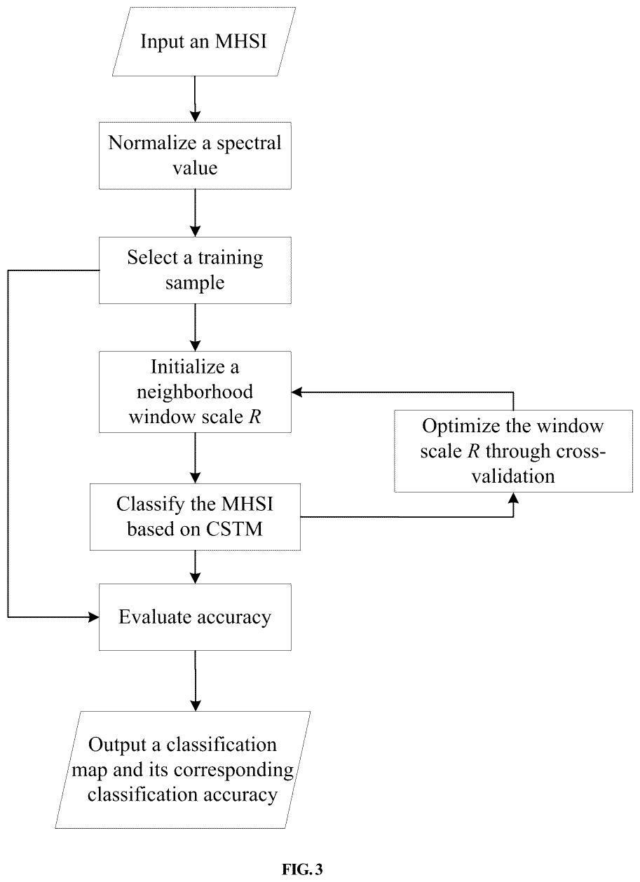

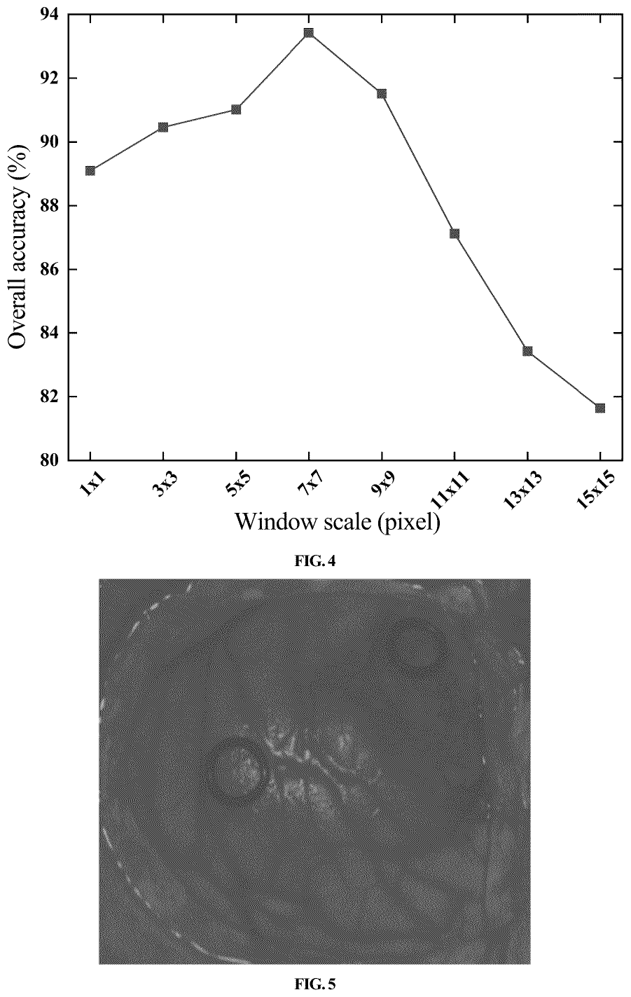

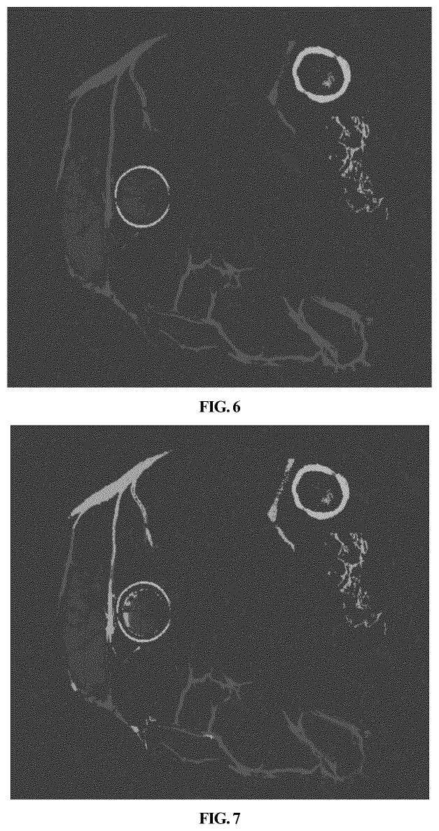

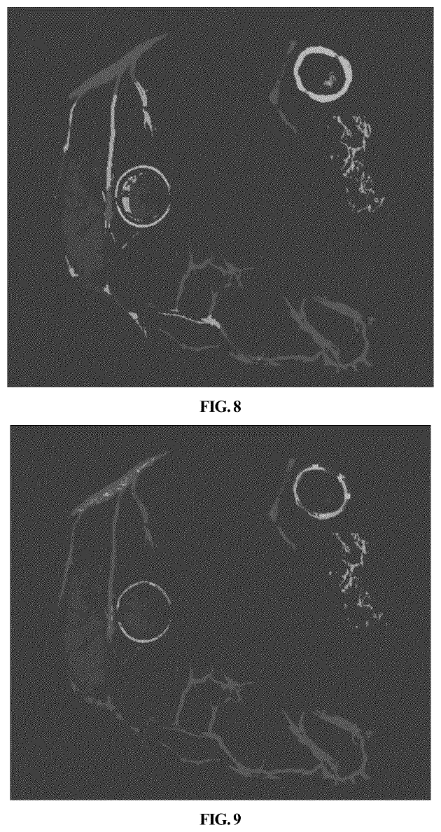

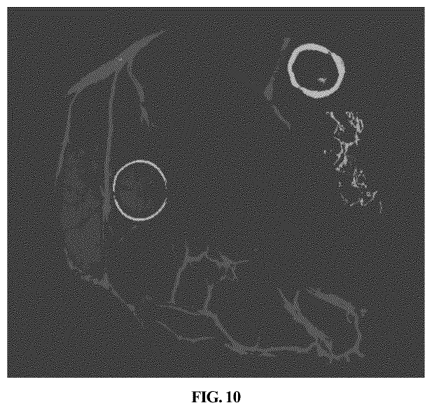

As shown in FIG. 3 to FIG. 10, the fast MHSI classification method based on similarity tangent mapping in the embodiments of the present disclosure is applied to classification of an HSI dataset of an In-Vivo human brain. The HSI contains 826 bands, 127 noise bands are removed, and 699 bands are retained. The HSI has a size of 443×479 and a spatial resolution of 128.7 μm. A real label map mainly contains three tissue categories and one type of background. Quantities of labeled pixels for different categories are shown in Table 1. Results of different algorithms are shown in FIG. 6 to FIG. 10.

TABLE 1
Quantity of label samples

| | | Quantity of pixels allocated to each label | | | | |
|---|---|---|---|---|---|---|
| | | Normal | Tumor | Vascular | | |
| HSI | All Pixels | tissue | tissue | tissue | Background | Total |
| 12-01 | 443 × 479 | 4516 | 855 | 8697 | 1685 | 15753 |

As shown in Table 2, compared with SVM, JNN, and JSRC algorithms, the method in the present disclosure can achieve higher classification accuracy, stability, and operational efficiency.

TABLE 2
Classification accuracy and computation time of CSTM classification and different methods

| | Method | | | | | | |
|---|---|---|---|---|---|---|---|
| Evaluation indicator | SVM | JNN | | JSR | | CSTM | |
| Kappa coefficient | 0.6655 (0.1271) | 0.7543 | (0.0809) | 0.7900 | (0.0575) | 0.8990 | (0.0447) |
| Overall accuracy (%) | 76.84 (10.35) | 83.84 | (5.69) | 87.25 | (3.48) | 93.76 | (3.01) |

| | | | | |
|---|---|---|---|---|
| Running time (s) | 2.629 | 0.876 | 16.26 | 0.226 |

Although the embodiments of the present disclosure are described with reference to the accompanying drawings, those skilled in the art may make various modifications and variations without departing from the spirit and scope of the present disclosure. These modifications and variations shall fall within the scope defined by the claims.

1. A fast medical hyperspectral image (MHSI) classification method based on similarity tangent mapping, comprising:
preprocessing a to-be-classified MHSI;
extracting a sample set from the to-be-classified MHSI;
dividing the sample set into a training sample set and a test sample set;
constructing a cosine similarity tangent mapping (CSTM) model based on the training sample set; and
inputting the test sample set into the CSTM model to obtain a classification result.

2. The fast MHSI classification method based on similarity tangent mapping according to claim 1, wherein the preprocessing a to-be-classified MHSI comprises:
obtaining a pixel quantity, a spectral dimension, and a tissue category of the to-be-classified MHSI; and
normalizing, band by band, a spectral value corresponding to each pixel.

3. The fast MHSI classification method based on similarity tangent mapping according to claim 1, wherein the extracting a sample set from the to-be-classified MHSI comprises:
extracting a sample quantity and a tissue category label of each sample.

4. The fast MHSI classification method based on similarity tangent mapping according to claim 1, wherein the constructing a CSTM model based on the training sample set comprises:
calculating a cosine similarity between a to-be-classified pixel and each training sample in feature space to constitute a cosine similarity matrix;
performing tangent mapping for the cosine similarity matrix;
calculating a similarity between the to-be-classified pixel and a training sample of each different tissue category by combining a spatial neighborhood; and
allocating a label to the to-be-classified pixel based on a highest similarity.

5. The fast MHSI classification method based on similarity tangent mapping according to claim 4, wherein the calculating a similarity between the to-be-classified pixel and a training sample of each different tissue category by combining a spatial neighborhood comprises:
obtaining a pixel and a neighborhood scale within the spatial neighborhood of the to-be-classified pixel;
obtaining a highest similarity between the to-be-classified pixel and each type of sample based on the cosine similarity matrix;
representing a similarity between the to-be-classified pixel and each type of sample by using an average similarity of all neighborhood pixels; and
comparing similarities between the to-be-classified pixel and different tissue categories, and adding the to-be-classified pixel to a tissue category with a highest similarity.

6. The fast MHSI classification method based on similarity tangent mapping according to claim 5, wherein the obtaining a pixel and a neighborhood scale within the spatial neighborhood of the to-be-classified pixel comprises:
obtaining an optimal neighborhood window scale through k iterative cross-validation operations.

7. The fast MHSI classification method based on similarity tangent mapping according to claim 2, wherein the normalizing, band by band, a spectral value corresponding to each pixel comprises:
obtaining a first difference between the pixel and a minimum spectral value of a corresponding band;
obtaining a second difference between a maximum spectral value of the corresponding band of the pixel and the minimum spectral value; and
normalizing the spectral value of the pixel into a ratio of the first difference to the second difference.

8. The fast MHSI classification method based on similarity tangent mapping according to claim 6, wherein the obtaining an optimal neighborhood window scale through k iterative cross-validation operations comprises:
randomly dividing training sample data into k equal fractions;
taking turns to use k−1 fractions of data as training data and remaining one fraction of data as test data;
taking an average value of iterative crossover results as estimation accuracy; and
taking a neighborhood window scale corresponding to highest accuracy as the optimal neighborhood window scale.
